(19) United States
(12) Patent Application Publication
(10) Pub. No.: US 2006/0173427 A1
(43) Pub. Date: Aug. 3, 2006

(54) Post operative breast surgery drain support garment

(76) Inventors: Edmundo L. Urbina, West Cape May, NJ (US); Lucille R. Urbina, West Cape May, NJ (US); Angela A. Pizzo, Cherry Hill, NJ (US); Joanne M. Pizzo, Cherry Hill, NJ (US); Joseph A. Pizzo SR., Cherry Hill, NJ (US)

Correspondence Address: Cheryl F. Cohen LLC, 2409 Church Road, Cherry Hill, NJ, 08002, US

(21) Appl. No.: 11/050,365

(22) Filed: Feb. 3, 2005

(51) Int. Cl. A61M 1/00

(52) U.S. Cl. 604/327

(57) ABSTRACT

A post operative breast surgery drain support garment including a form fitting support structure and one or more pouches attached to the front of the support structure and unobstructed from access. Each pouch is adapted in size and shape so as to form fittingly receive and maintain therein a single drainage receptacle. A drainage tube passes through drainage tube openings defined in the garment. Retaining devices are releasably mounted to the support structure and extend across at least a portion of the drainage tube openings to retain the drainage tube against the body thereby minimizing uncomfortable tugging. Retaining devices preferably include two main releasable retaining devices disposed substantially horizontal and substantially parallel to one another. Additional restraint may be provided by a supplemental retaining device disposed substantially perpendicular to the first and second main releasable retaining devices. The retaining devices substantially inhibit movement of the drainage tube thereby minimizing uncomfortable tugging of the drainage tube at the point of exit from the body.

### BACKGROUND OF THE INVENTION

1. Field of the Invention

The present invention is directed to a garment and, in particular, to a garment to be worn during post operative recovery after breast surgery that supports a drainage receptacle while restricting movement and tugging of the associated drainage tube at the point of exit from the body.

2. Description of Related Art

The occurrence of breast cancer in both men and women is on the rise. Conventional treatment for this disease often includes surgery such as a lumpectomy, mastectomy and/or reconstructive breast surgery. Depending on the spread of the disease, surgery may include removal of some, if not all, of the breast tissue and lymph nodes associated with one or both breasts. During surgery a drain (e.g., Jackson-Pratt drain or Snyder drain) is surgically implanted proximate each operative area to draw off any fluid (e.g., blood and/or lymph node fluid) from around the surgical site thereby reducing the chance of infection while promoting healing. Accordingly, post operative recovery may require at least one or as many as four separate drains depending on the following factors: (i) whether surgery was performed in one or two areas (e.g., breast tissue and/or lymph nodes) and (ii) whether the operation involved one or both breasts. Each drain includes several feet of flexible tube (e.g., plastic or rubber tube) that carries the fluid from the surgical site out of the body and into a bulbous drainage receptacle or reservoir. The drainage tube is secured in place via sutures at the point of exit from the patient's body and remains in place until the patient's body is able to reabsorb the fluid on its own.

Typically the drain remains implanted for several weeks after surgery. Accordingly, the patient must be able to provide daily maintenance of the drain, preferably without the help of another. Maintenance includes periodically emptying the bulbous drainage receptacle, preferably at least every 8 hours. In so doing, the patient removes a plug or closure and empties the contents of the drainage receptacle into a measuring container provided. It is important for the patient to monitor and notify their physician of the quantity of fluid dispensed from the drainage receptacle. After the fluid has been evacuated from the drainage receptacle, the bulb is squeezed in one hand (creating a vacuum pressure inside the drainage receptacle) while replacing the plug with the other hand. The negative pressure within the drainage receptacle draws the fluid from the surgical site.

Patients recovering from surgery often complain that, despite all efforts, dangling of the weighted drainage receptacle and drainage tube during routine daily activities tugs on the tube at the point of suture causing much discomfort. In the case in which the tube is subject to a severe tug it is possible that the sutures may give way dislodging the drain and requiring medical attention. Heretofore, to prevent dislodgement patients pinned the drainage tube directly to the hospital gown thereby limiting some but not all of its movement. Pinning does not sufficiently restrict displacement of the drainage tube at the point of exit from the body to eliminate the problem. Another solution more restrictive then pins to substantially prevent displacement and/or dislodgement is to tape the drainage tube directly to the body. Tape must be removed at least once per day to allow for bathing. Sores and/or irritation to the skin as well as great discomfort is experienced from frequent removal of the tape. Moreover, the use of tape does not afford the desired freedom of movement in that the patient is in constant fear of the dislodgement of the tube from the exit site of the body. When multiple drainage tubes are utilized, the mental stress and physical discomfort from removal and reapplication of the multiple strips of tape is intensified.

Pockets or pouches have been employed in garments to support drainage receptacles during post operative recovery. Some garments are bulky, loose fitting housecoats having large pockets in which the patient stores the drainage receptacle and tube. These housecoat garments due to their bulky size and shape do little, if anything, to prevent swinging of the drainage receptacle and tugging on the drainage tube. Camisoles have also been modified or adapted to include detachable pockets for housing the drainage receptacle and associated tube. These camisoles are clearly more form fitting than their predecessor housecoats and thus restrict swinging of the drainage receptacle. Nevertheless, these garments are problematic in that they must be stepped into through the neck opening thereby requiring that the garment be pulled up over the surgical areas with the potential for dislodging a suture or the drainage tube. Such camisoles employ removable drainage pouches mounted to a bra like support structure and hidden or obstructed by a camisole portion that extends over the torso to conceal from view the pouches. To obtain a clear unobstructed view and gain access to the drainage receptacles during routine maintenance, the patient must raise the camisole portion upwards over their breasts. Raising of ones arms in such an upwards movement may cause discomfort to the patient not to mention the additional pressure caused by the excess camisole fabric when gathered upwards against the surgical area proximate the breast. In addition, like the housecoat, the camisole has no feature for limiting or restricting movement of the drainage tube itself proximate the point of exit from the body.

It is therefore desirable to develop a garment to be worn during recovery for post operative breast surgery that solves the aforementioned problems by substantially prohibiting dislodgement and/or displacement of the drainage tube proximate the point of exit from the body without discomfort to the patient while remaining unobstructed and completely accessible by the patient so that they can independently provide all routine daily care and maintenance of the device.

### SUMMARY OF THE INVENTION

An object of the present invention is to design a post operative breast surgery drain support garment that eliminates the need for pins or tape.

Another object of the invention is to design a post operative breast surgery drain support garment that supports a drainage receptacle.

Still another object of the invention is to develop a post operative breast surgery drain support garment that minimizes movement of the drainage tube proximate the point of suture to the body.

The present invention is directed to a support garment including a form fitting support structure having a front, a back, and sides. One or more pouches are attached to the front of the support structure and unobstructed from access. Each pouch is adapted in size and shape so as to form fittingly receive and maintain therein a single drainage receptacle. When the garment is worn the pouch is disposed below a patient's heart. A releasable closure (e.g., zipper) is positioned proximate the front of the support structure to minimize arm movement while dressing and during removal. A drainage tube is passed through a drainage tube opening defined in the support structure. Retaining devices are releasably mounted to the support structure to retain the drainage tube against the body thereby minimizing uncomfortable tugging. Preferably, retaining devices include two main releasable retaining devices disposed substantially horizontal and substantially parallel to one another extending across at least a portion of the drainage tube opening. The drainage tube is bound beneath the first releasable retaining device, extends upwards and then passes downwards over the first releasable retaining device, and then is restrained beneath the second releasable retaining device. Additional restraint may be provided by a supplemental retaining device disposed substantially perpendicular to the first and second main releasable retaining devices. The drainage tube being restricted beneath the supplemental retaining device and above one of the first or second main releasable retaining devices. The retaining devices substantially inhibit movement of the drainage tube thereby minimizing uncomfortable tugging of the drainage tube proximate the point of exit from the body.

The present invention is also directed to a method for using the post operative breast surgery drain support garment described above. Initially, the drainage tube is arranged beneath the first main releasable retaining device in a direction substantially opposite a force imposed by gravity. The reasoning for directing the drainage tube in an upwards direction against the force imposed by gravity is to reduce tugging of the drainage tube proximate the point of suture to the body. Then the drainage tube is passed over the first main releasable retaining device beneath which it was secured. Next, the drainage tube is bound beneath the second main releasable retaining device. Finally, the drainage tube is restrained between one of the first and second main releasable retaining devices and the supplemental releasable retaining device.

### BRIEF DESCRIPTION OF THE DRAWING

The foregoing and other features of the present invention will be more readily apparent from the following detailed description and drawings of illustrative embodiments of the invention wherein like reference numbers refer to similar elements throughout the several views and in which:

FIG. 1 is a front view of an exemplary post operative breast surgery drain support garment in accordance with the present invention while the releasable closure is in a closed state and worn by a patient;

FIG. 2 is a rear view of the garment of FIG. 1 while the releasable closure is in a closed state;

FIG. 3a is a left side view of the garment of FIG. 1 while the releasable closure is in an open state;

FIG. 3b is a right side view of the garment of FIG. 1 while the releasable closure is in an open state and accommodating two drains;

FIG. 4 is an exterior view of the garment of FIG. 1 laid out flat while the releasable closure is in an open state; and

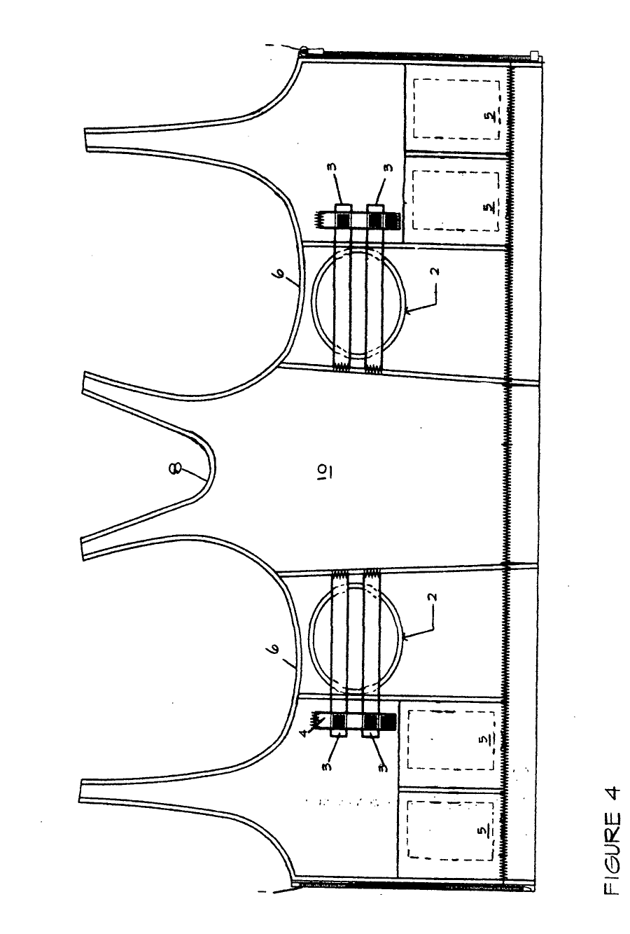

FIG. 5 is partial cross-sectional view of the drainage tube releasable retaining device of FIG. 1.

### DETAILED DESCRIPTION OF THE INVENTION

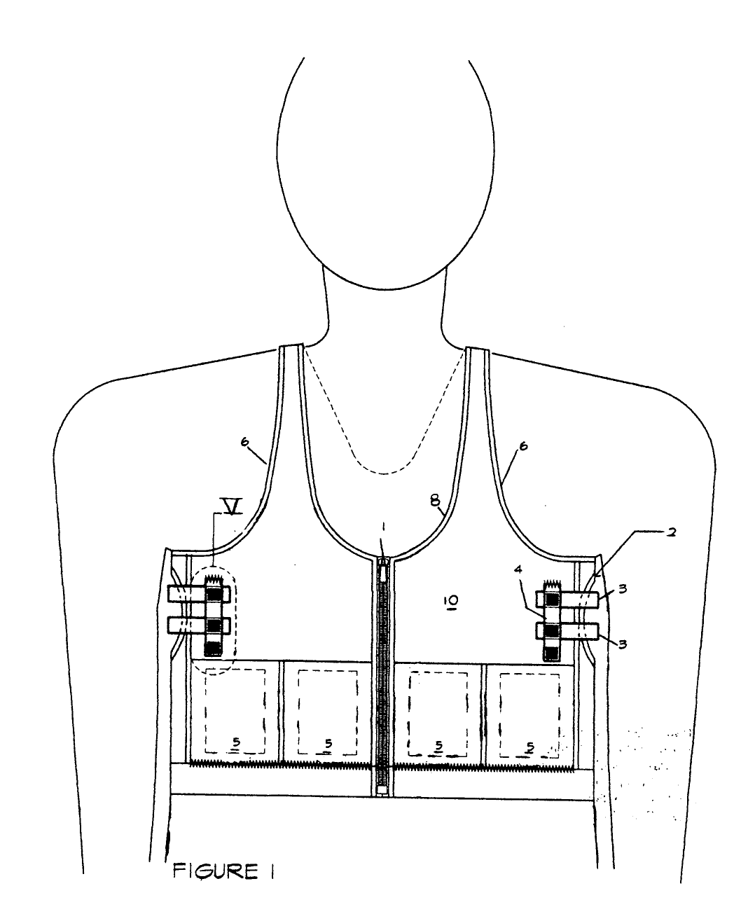

For purposes of illustration only, the present inventive post operative breast surgery drain support garment is formed from a support structure 10 shown and described as being bra-like in appearance. The overall shape of the support structure may be modified both aesthetically and ergonomically, to accommodate the needs of either men or women. Preferably, the support structure is made of a soft comfortable seamless fabric that is form fitting to be retained proximate, most preferably in direct contact with, the body. Elastic may be disposed about at least some portion of its perimeter to retain the support structure 10 relatively close to the body. Although shown extending just below the breasts, the design of the support structure may be varied to extend further down the torso of the body (more closely resembling a tank top or undershirt, preferable to men). In FIG. 1 the support structure 10 is shown with a butterfly back. The positioning of the straps is not critical to the invention and over the shoulder straps may be used instead. Straps are preferably adjustable to fit the particular individual. A releasable closure 1 (e.g., a zipper, VELCRO™, hooks and eyes, snaps, buttons, or buckles) is positioned in the front of the garment between the breasts so that the patient may readily dress and undress with minimal arm movement. A front closure avoids the extreme discomfort of the patient having to reach the rear of the garment. The releasable closure 1 is positionable between one of two states, i.e., a released or open state, otherwise, the closure is in a secured or closed state. The support structure 10 has a neck or head opening 8 and two armhole openings 6. Each armhole opening 6 is sufficient in size so that the patient can readily get in and out of the garment with minimal arm movement. Drainage tube openings 2 are provide and sized so as to receive therethrough a drainage tube 7 and not interfere with any of the surgical sites. In a preferred embodiment, a single relatively large drainage tube opening 2 is defined in each side of the support structure 10, as shown in FIG. 1. Alternatively, multiple smaller sized drainage tube openings 2 may be defined on each side of the support structure one for each surgical site. For example, in the case of two drains associated with a single breast either one large or two smaller sized drainage tube openings 2 may be utilized. Once again, regardless of the quantity or size of the drainage tube openings, they must be of sufficient size and/or number so as not to interfere with any of the surgical sites.

One or more pouches 5 are secured to the support structure 10, preferably two pouches on either side of the releasable closure 1. When assembling the garment, each pouch 5 must be disposed so that the entire drainage receptacle 9 when housed therein is positioned below the patient's heart to ensure that the fluid is carried away from the patient's heart. The pouches 5 preferably are permanent secured (e.g., sewn) onto the support structure 10. Alternatively, the pouches may be releasably secured to the support structure 10, for example, via zippers, VELCRO™, hooks and eyes, snaps, buttons, or buckles. If the pouch 5 is detachable then it may be positioned as desired on the support structure 10 and if not being utilized may be readily removed so as not to be cumbersome and in the way. One advantage associated with the pouch 5 being permanently secured to the support structure is that it ensures that the drainage receptacle 9 when inserted therein will always be disposed below the patient's heart. Each pouch 5 is preferably adapted in size to snugly friction or form fit a single drainage receptacle. The overall configuration may be modified so that each pouch 5 is sized to accommodate more than one drainage receptacle 9 thereby reducing the number of pouches. Since the pouches are preferably dimensioned so as to snugly fit a single drainage receptacle there is little chance of it falling out. However, if the size of the pouch becomes to large or multiple drainage receptacles are retained in a single pouch 5 then a top closure such as a zipper, VELCRO™, hook and eye, snap, button, or buckle may be utilized to substantially prevent the drainage receptacle from falling out when the patient bends over.

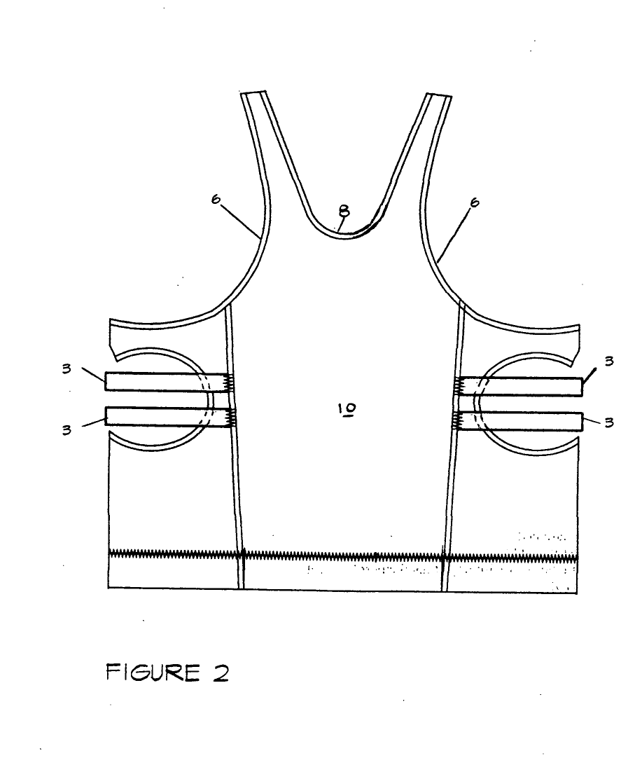

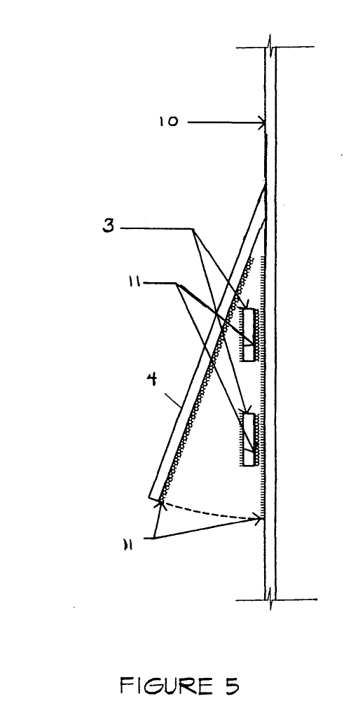

As mentioned one of the most substantial causes of discomfort and anxiety to the patient during post operative recovery is associated with the weight and tugging of the drainage tube 7 on the suture site. In order to reduce, if not substantially prevent, tugging on the drainage tube, a plurality of releasable retaining devices 3, 4, e.g., straps or flaps, are secured across at least a portion of each drainage tube opening 2, as shown in the rear and side view of FIGS. 2 & 3a, respectively. The garment preferably employs two main releasable retaining devices 3 disposed substantially parallel to one another and extends substantially horizontally across at least portion of each drainage tube opening 2. A single supplemental releasable retaining device 4 is disposed substantially perpendicular relative to that of the main releasable retaining devices 3. Preferably one end of the main releasable retaining device 3 is permanently secured to the drain support garment 10, while the opposite free end is releasably secured to the garment via a releasable securing device 11 such as a zipper, VELCRO™, hook and eye, snap, button, or buckle. As with all aspects of the present invention it is desirable to make the patient as self-sufficient as possible in the care and maintenance of the drain system with little, if any, assistance required from others while optimizing comfort. In keeping with this goal the fixed end of each of the main releasable retaining devices 3 is preferably secured to the back of the support structure 10, while the releasable free end is disposed towards the front so as to be easily engaged/disengaged by the patient. Instead of one end being permanently secured, both ends of the main retaining devices 3 may alternatively be releasably secured to the support structure 10. This modified design, however, is not preferred since the end disposed proximate the back of the bra may become disengaged causing great discomfort to the patient in having to reach around to the back to reposition it in place. The supplemental releasable retaining device 4 preferably has one fixed or secured end and multiple points of releasable secured engagement (e.g., VELCRO™, hook and eye, snap). In the preferred embodiment, as shown in the partial cross-sectional view of FIG. 5, the points of releasable secured engagement are disposed one proximate the releasably engaged free end of each main releasable retaining device 3 and the other engaging the free end of the supplemental releasable retaining device 4 directly to the support structure 10 itself. Securing the supplemental releasable retaining device 4 directly atop the free ends of the main releasable retaining device 3 helps ensure that they remain engaged. Any number of one or more points of secured engagement may be selected, however, the greater the number of points of releasably secured engagement and/or the shorter the distance between any two adjacent points of engagement the more restricted movement of the drainage tube 7. The quantity and positioning of each of the releasable retaining devices 3, 4 may be varied, as desired, to include any number of one or more in any desired configuration. Furthermore, the garment may eliminate all together either the main releasable retaining devices 3 or the supplemental releasable retaining devices 4, but preferably not both. A substantially perpendicular arrangement relative to one another is preferred, but other configurations of the releasable retaining devices 3, 4 are contemplated and within the intended scope of the invention.

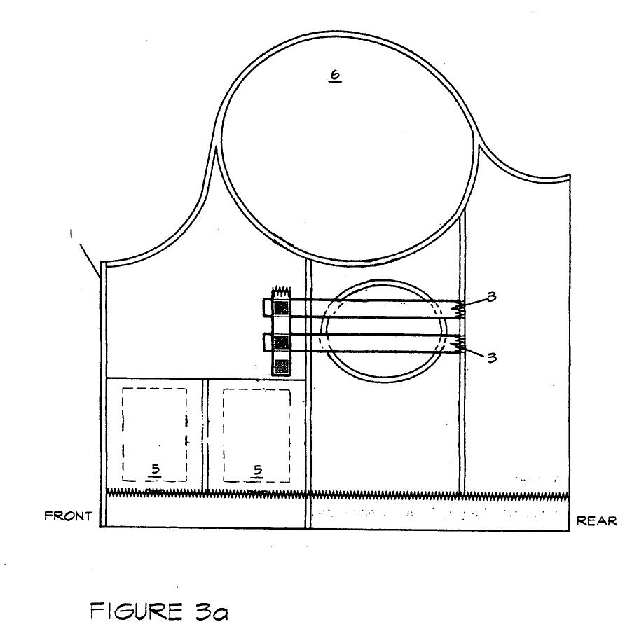

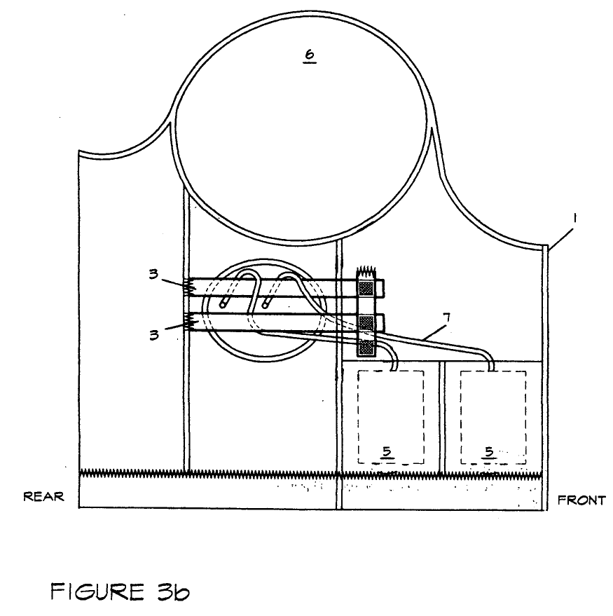

Initially, the releasable closure 1 is disengaged and the drain support garment 10 is opened whereby the patient inserts their arms into the respective armholes 6 much like a vest so that the closure is positioned between the breasts. In this position, the patient is able to get dressed without having to pull the garment 10 over their head and engage the closure 1 from the front with minimal arm movement. Each drain receptacle 9 is received in their corresponding pouch 5. Next the drainage tube 7 associated with each drain is woven between the main releasable retaining devices 3 to retain them in direct contact with the patient's body. In the side view of FIG. 3b, the drainage tube 7 is secured in direct contact with the side of the patient's body by the main releasable retaining devices 3. Specifically, the drainage tube exits from the body and extends upwards towards the shoulders while in contact with the patient's body. It is preferred for the drainage tube 7 to first extend upwards to remove any drag, pulling or tugging directly at the exit site. A first main releasable retaining device 3 is placed over the drainage tube 7 and secured in place (e.g., by engaging the VELCO) immobilizing the drainage tube in an upwards pointing direction against the body proximate its point of suture or exit. The drainage tube 7 then passes over the first main releasable retaining device 3 and once again is placed against the patient's body beneath a second main releasable retaining device 3 that is anchored into an engaged position. Movement and tugging of the drainage tube 7 is severely restricted or limited by the main releasable retaining devices 3. Additional restraint against movement or tugging may be provided by fastening the drainage tube 7 between adjacent points of engagement of the supplemental releasable retaining device 4 positioned proximate the front of the support structure. More releasable straps may be provided to further restrict mobility of the drainage tube 7.

The present inventive drain support garment 10 has been shown and described as having two drains associated with each breast requiring a total of four separate tubes and corresponding drainage receptacles. It is, however, contemplated and within the intended scope of the present invention to modify the bra so that the number of drains is less than four but at least one and that the bra has at least the same number of pouches as drains. More pockets can be provided than drains so that the garment may be universally used under any post operative breast surgery situation. Otherwise, the garment may be modified in design to specifically accommodate any number one or more drains so that the number of drains and the number of pouches is the same. Furthermore, the releasable retaining devices may be a single strap or multiple straps in any arrangement, so long as the drainage tube may be releasably bound beneath one or more retaining devices proximate the point of suture or exit from the body. This invention therefore substantially prohibits uncomfortable tugging and possible dislodgement of the drainage tube at the point of suture or exit from the body, while promoting patient mobility and independence without sacrificing comfort or peace of mind.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. A post operative breast surgery drain support garment comprising:
a form fitting support structure having a front, a back, and sides;
at least one pouch attached to the front of the support structure and unobstructed from access, each of the at least one pouch being adapted in size and shape so as to form fittingly receive and maintain therein a single drainage receptacle, when the garment is worn the at least one pouch is disposed below a patient's heart.

2. The garment in accordance with claim 1, further comprising a releasable closure disposed in the front of the support structure.

3. The garment in accordance with claim 1, further comprising at least one releasable retaining device having a first end and an opposite second end, the first and second ends of the at least one releasable retaining device being secured to the support structure, the at least one releasable retaining device being adapted to restrain a drainage tube.

4. The garment in accordance with claim 1, further comprising at least one drainage tube opening disposed proximate the side of the support structure adapted in size for receiving therethrough a drainage tube.

5. The garment in accordance with claim 4, further comprising at least one releasable retaining device extending across at least a portion of the at least one drainage tube opening, the at least one releasable retaining device having a first end and an opposite second end, the first and second ends of the at least one releasable retaining device being secured to the support structure.

6. The garment in accordance with claim 1, wherein the at least one pouch is adapted in size and shape to receive and maintain the single drainage receptacle in a substantially upright direction.

7. The garment in accordance with claim 1, wherein the at least one pouch having an opening and being secured about its entire perimeter, except for at its opening, to the support structure.

8. A post operative breast surgery drain support garment comprising:
a support structure having defined therein a head opening, a torso opening, two arm openings and at least one drainage tube opening adapted in size to receive therethrough at least one drainage tube.

9. The garment in accordance with claim 8, wherein the support structure has a front, a back and two sides, and wherein the at least one drainage tube opening is disposed proximate the side of the support structure.

10. The garment in accordance with claim 9, wherein the support structure has two drainage tube openings defined therein, one proximate each side of the support structure.

11. The garment in accordance with claim 10, wherein the support structure has four drainage tube openings defined therein, two proximate each side of the support structure.

12. The garment in accordance with claim 9, further comprising at least one releasable retaining device disposed to extend across at least a portion of the at least one drainage tube opening.

13. The garment in accordance with claim 12, wherein the at least one releasable retaining device is a strap having a first end and an opposite second end that is releasably secured proximate the front of the support structure.

14. The garment in accordance with claim 13, wherein the first end of the at least one releasable retaining device is permanently secured proximate the back of the support structure.

15. The garment in accordance with claim 12, wherein the garment comprises at least two releasable retaining devices disposed substantially parallel to one another.

16. The garment in accordance with claim 15, wherein the at least two releasable retaining devices comprises at least one main releasable retaining device arranged substantially perpendicular to at least one supplemental releasable retaining device.

17. The garment in accordance with claim 16, wherein the at least one supplemental releasable retaining device is secured across the at least two main releasable retaining devices.

18. The garment in accordance with claim 12, wherein the at least one releasable retaining device comprises two main releasable retaining devices disposed proximate the side of the support structure and extending across at least a portion of the at least one drainage tube opening.

19. The garment in accordance with claim 18, wherein the at least two main releasable retaining devices are disposed substantially parallel to one another when secured to the support structure.

20. The garment in accordance with claim 19, wherein the two main releasable retaining device are disposed substantially horizontally when secured to the support structure.

21. The garment in accordance with claim 1, wherein the support structure is in the shape of a bra or tank shirt.

22. A method for supporting a drain in a garment after post operative breast surgery, the garment including a support structure, at least one drainage tube opening defined in the support structure and adapted for receiving therethrough at least one drainage tube, at least one releasable retaining device extending across at least a portion of the drainage tube opening, comprising the step of:
threading the drainage tube through the drainage tube opening; and
securing the drainage tube beneath the at least one main releasable retaining device.

23. The method in accordance with claim 22, wherein the at least one releasable retaining device comprises a main releasable retaining device and the securing step comprises the steps of:
arranging the drainage tube beneath the main releasable retaining device in a direction substantially opposite a force imposed by gravity; and
passing the drainage tube over the main releasable retaining device beneath which it was secured.

24. The method in accordance with claim 22, wherein the at least one releasable retaining device comprises a first main releasable retaining device, a second main releasable retaining device disposed substantially parallel to the first main releasable retaining device, and a supplemental releasable retaining device; the securing step comprising the steps of:
arranging the drainage tube beneath the first main releasable retaining device in a direction substantially opposite a force imposed by gravity;
passing the drainage tube over the first main releasable retaining device beneath which it was secured;
binding the drainage tube beneath the second main releasable retaining device; and
restraining the drainage tube between one of the first and second main releasable retaining devices and the supplemental releasable retaining device.

25. The method in accordance with claim 23, wherein the arranging step includes directing the drainage tube upwards when passed beneath the main releasable retaining device to reduce tugging of the drainage tube.
